(19) United States
(12) Patent Application Publication
(10) Pub. No.: US 2018/0297842 A1
(43) Pub. Date: Oct. 18, 2018

(54) METHOD OF GENERATING HYDROGEN-CONTAINING LIQUID

(71) Applicant: MiZ Company Limited, Kanagawa (JP)

(72) Inventor: Ryousuke KUROKAWA, Kanagawa (JP)

(21) Appl. No.: 15/787,052

(22) Filed: Oct. 18, 2017

(30) Foreign Application Priority Data
Oct. 27, 2016 (JP) 2016-210172

(51) Int. Cl. C01B 3/08; C01B 3/06; A61K 33/00

(52) U.S. Cl. CPC C01B 3/08; A61K 33/00; C01B 3/065

(57) ABSTRACT

A method is provided, comprising: preparing a hydrogen generating agent (14) that reacts with water to generate hydrogen gas, a first container (11) that encloses a liquid (16) applicable to a living organism and has hydrogen gas permeability, and a second container (12) that has hydrogen gas permeability equal to or lower than the hydrogen permeability of the first container; storing the first container and the hydrogen generating agent in the second container and sealing the second container with no water except moisture in air; maintaining the second container storing the first container and the hydrogen generating agent in a frozen state from when the second container is sealed until when the liquid is used; upon use of the liquid, placing the second container storing the first container and the hydrogen generating agent in an unfreezing atmosphere so that the hydrogen generating agent reacts with water in the second container to generate hydrogen gas in the second container, the water in the second container including water vapor resulting from vaporization of water contained in the liquid stored in the first container and permeation of the water vapor through the first container into the second container and/or the moisture in air in the second container; and allowing the hydrogen gas generated in the second container to pass through the first container so that hydrogen molecules are contained in the liquid stored in the first container.

### BACKGROUND OF THE INVENTION

### 1. Technical Field of the Invention

The present invention relates to a method of generating hydrogen-containing liquid.

### 2. Description of the Related Art

The present applicants have previously proposed a method for producing a living organism-applicable hydrogen-contained fluid as a method of generating hydrogen-containing liquid. The method for producing a living organism-applicable hydrogen-contained fluid includes supplying hydrogen molecules to a living organism-applicable fluid stored in a container having water vapor permeability. Specifically, the method includes a step of enclosing the container and a hydrogen generation system located outside the container in a wrapping material having low hydrogen molecule permeability, a step of increasing humidity in a space between the container and the wrapping material, and a step of transmitting hydrogen molecules generated by the hydrogen generation system from outside the container into the living organism-applicable fluid (Patent Document 1: JP5935954B).

Patent Document 1: JP5935954B

In the above conventional production method, however, generation of hydrogen gas is initiated from when enclosing the container and the hydrogen generation system in the wrapping material and, accordingly, transportation and storage of the wrapping material are performed in a state in which the wrapping material is filled with hydrogen gas. The conventional method may thus be more improved in the safety during the course of distribution of products and/or during the storage period and improved in reduction of the amount of hydrogen gas.

An object of the present invention is therefore to provide a method of generating hydrogen-containing liquid which has high safety and with which hydrogen molecules do not substantially decrease.

### SUMMARY OF THE INVENTION

The present invention achieves the above object through
preparing a hydrogen generating agent that reacts with water to generate hydrogen gas, a first container that encloses a liquid applicable to a living organism and has hydrogen gas permeability, and a second container that has hydrogen gas permeability equal to or lower than the hydrogen permeability of the first container;
storing the first container and the hydrogen generating agent in the second container and sealing the second container with no water except moisture in air;
maintaining the second container storing the first container and the hydrogen generating agent in a frozen state from when the second container is sealed until when the liquid is used;
upon use of the liquid, placing the second container storing the first container and the hydrogen generating agent in an unfreezing atmosphere so that the hydrogen generating agent reacts with water in the second container to generate hydrogen gas in the second container, the water in the second container including water vapor resulting from vaporization of water contained in the liquid stored in the first container and permeation of the water vapor through the first container into the second container and/or the moisture in air in the second container; and
allowing the hydrogen gas generated in the second container to pass through the first container so that hydrogen molecules are contained in the liquid stored in the first container.

In the above invention, it is more preferred that placing the second container in the unfreezing atmosphere include maintaining the second container in a temperature environment higher than room temperature for a certain period of time and it is also more preferred that the temperature environment higher than room temperature be 30° C. or higher.

According to the present invention, water that is reactive with the hydrogen generating agent is maintained in a solidified state or the second container storing the hydrogen generating agent is maintained in a frozen state from when the second container is sealed until when the liquid is used. Hydrogen gas is therefore not generated during the course of distribution of products and during the storage period, and hydrogen gas is generated only when the liquid is used. Thus, a method of generating hydrogen-containing liquid can be provided which has high safety and with which hydrogen molecules do not substantially decrease.

### BRIEF DESCRIPTION OF DRAWINGS

FIG. 1 is a flow diagram illustrating an embodiment of the method of generating hydrogen-containing liquid according to the present invention; and

FIG. 2 is a flow diagram illustrating another embodiment of the method of generating hydrogen-containing liquid according to the present invention.

### DESCRIPTION OF THE PREFERRED EMBODIMENT

### First Embodiment

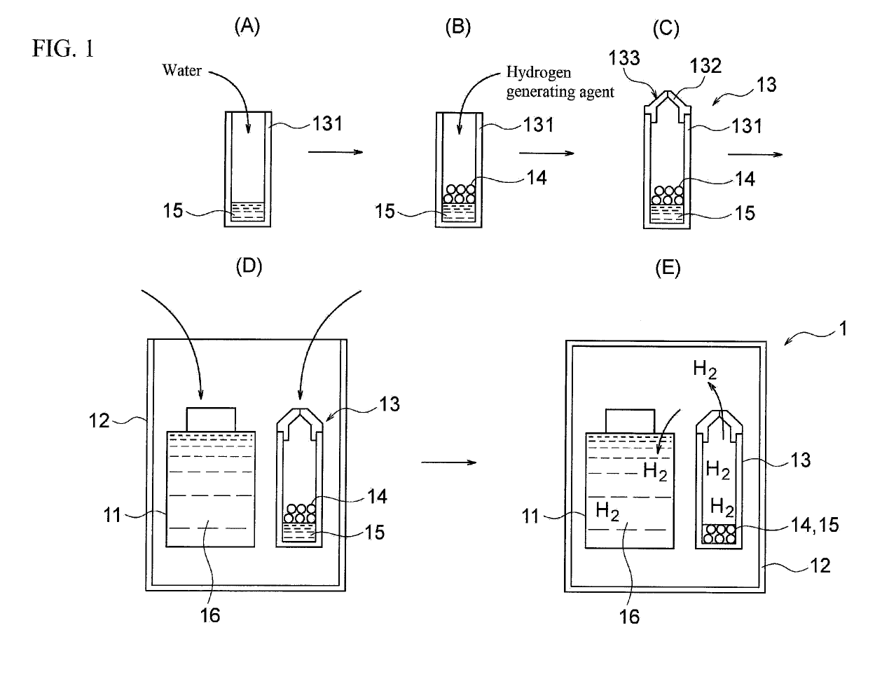

FIG. 1 is a flow diagram illustrating an embodiment of the method of generating hydrogen-containing liquid according to the present invention. This method includes preparing a hydrogen generating agent 14 that reacts with water to generate hydrogen gas, a first container 11 that encloses a liquid 16 applicable to a living organism and has hydrogen gas permeability, a second container 12 that has hydrogen permeability equal to or lower than the hydrogen permeability of the first container 11, and a third container 13 that can store the hydrogen generating agent 14 and water 15 and discharge hydrogen gas generated inside to outside. These first container 11, second container 12, third container 13, hydrogen generating agent 14, water 15, and liquid 16 constitute a device 1 for generating hydrogen-containing liquid according to the present embodiment.

Examples of the liquid 16 applicable to a living organism (human and other animals) include normal saline solution prepared in terms of osmolality for use as injection, intravenous drip, transfusion, and the like; liquid for injection and oral liquid for resupply of water, nutrition, electrolytes, and the like; liquid for injection and normal saline solution in which a medical agent is dissolved; liquid medical agent; blood preparation (blood for blood transfusion) and own blood to be used for blood transfusion; enteral solution; and drinking water. Examples of the liquid 16 further include organ preservative solution prepared to preserve organs; cell culture liquid; cell maintenance liquid; liquids including lymph cells and vaccines used in cancer immune therapy, vaccine therapy, and similar therapies; peritoneal dialysis solution; dialysis solution; and myocardial protective medicine. The living organism-applicable liquid 16 of the present embodiment refers to a concept that encompasses general liquids to be applied orally or parenterally to living organisms for the purpose of improvement in maintaining vital functions and prevention, treatment or the like of diseases and disorders.

By including hydrogen molecules in such a liquid 16 applicable to a living organism, it is possible to add functions and effects of hydrogen molecules for living organisms, such as an action of inhibiting oxidative stress, to the functions and effects which are intrinsically possessed by the liquid 16.

Diseases and disorders that can fall within the applicable region of liquids containing hydrogen molecules include, but are not limited to, liver damage due to chemicals and/or harmful substances; circulatory system diseases, such as ischemic reperfusion disorder and arteriosclerosis; digestive system diseases, such as gastric ulcer and gastric mucosal disorder; respiratory diseases; complications from diabetes (e.g. high blood pressure, cerebral infarction, myocardial infarction, etc.); renal diseases; cataract; skin diseases; various inflammatory diseases; neurological disorders; cancer; and oxidative stress diseases, such as aging, attributable to free radicals and/or lipid peroxide. In particular, liquids containing hydrogen molecules may be suitably applied to diseases related to acute oxidative stress, such as ischemic reperfusion disorder.

The liquid 16 of the present embodiment may contain a certain amount of hydrogen molecules prior to applying the device 1 for generating hydrogen-containing liquid according to the present embodiment. In this case, the device 1 for generating hydrogen-containing liquid according to the present embodiment is to complement hydrogen molecules to the liquid 16, and the concentration of dissolved hydrogen in the liquid 16 can be maintained for a long time.

It suffices that the first container 11 which encloses the above liquid 16 and has hydrogen gas permeability is a container (membrane) through which hydrogen molecules can pass. The first container 11 may preferably be, but is not limited to, a plastic container made of a material, such as polyethylene, polypropylene and polystyrene, which is used for infusion solution bags and intravenous bags. A glass container can also be used as the first container 11. In many cases, hydrogen molecules having a small molecular size can pass without problems even through containers characterized by oxygen gas barrier properties and/or water vapor barrier properties. The first container 11 for the liquid 16 applicable to a living organism may be further preferably treated with a process of controlling the permeation direction of hydrogen molecules to be irreversible so that hydrogen molecules can pass through the first container 11 by adsorption or separation, in particular, selectively pass through the first container 11 while the liquid 16 can stably maintain the hydrogen molecules which have once passed through the first container 11 and have been contained in the liquid 16. In a preferred embodiment, the first container 11 may be a translucent or transparent container through which the water level of the liquid 16 can be visually confirmed from external. This allows the user to confirm the consumption of the liquid 16 applicable to a living organism when using the liquid 16 for intravenous drip, etc.

In the present embodiment, hydrogen gas passes from outside through the first container 11 having hydrogen gas permeability, thereby to allow the hydrogen molecules to be contained in the liquid 16. Therefore the first container 11 may not have to be preliminarily opened, but the present invention does not exclude cases in which hydrogen molecules pass through the first container 11 in the opened state.

The first container 11 which encloses the above liquid 16 and has hydrogen gas permeability may be commercially available after the first container 11 has already been stored in one or more separate exterior bags. Even in such cases, the manufacturing method of the present embodiment is applicable, provided that the exterior bag or bags have hydrogen gas permeability. That is, the concept of the first container 11 of the present invention encompasses those in which the first container 11 is doubly or multiply covered with one or more separate exterior bags or the like, provided that the exterior bag or bags have hydrogen gas permeability.

The hydrogen generating agent 14 is a composition that reacts with liquid water or gaseous water (water vapor) to generate hydrogen. Liquid water and gaseous water will be collectively referred to as “water 15,” hereinafter. If necessary, the hydrogen generating agent 14 may contain an appropriate reaction promoter that promotes the hydrogen generation reaction. Examples of the hydrogen generating agent 14 include, but are not limited to, a metal having a higher ionization tendency than that of hydrogen, and hydrogenated compounds including a metal hydride. In consideration of good reactivity with water vapor (i.e. humidity), metal calcium, calcium hydride, metal magnesium, magnesium hydride, metal iron, and the like may preferably be used. In consideration of the safety of reaction products, metal magnesium, metal aluminum, metal zinc, metal nickel, metal cobalt, metal iron, and the like may be preferably used.

Examples of the hydrogen generation reaction promoter include a pH adjuster for adjusting pH to that suitable for promoting the reaction between the liquid water 15 or gaseous water 15 and the hydrogen generating agent 14. Examples of such a pH adjuster include substances that supply hydrogen ions (H⁺), such as citric acid, adipic acid, malic acid, acetic acid, succinic acid, gluconic acid, lactic acid, phosphoric acid, hydrochloric acid, sulfuric acid, and cation-exchange resin. When an amphoteric metal, such as aluminum and zinc, is used as the hydrogen generating agent 14, an alkaline agent, such as calcium hydroxide, calcium oxide, and anion-exchange resin, can also be used other than the above acids. Among these, calcium hydroxide (hydrated lime), burnt lime (calcium oxide), calcined calcium, magnesium oxide, magnesium hydroxide, anion-exchange resin, and the like may preferably be used.

Examples of the hydrogen generation reaction promoter also include a hygroscopic agent and a drying agent because such agents absorb humidity to promote the hydrogen generation reaction. Examples of the hygroscopic agent and drying agent include, but are not limited to, the above-described ion-exchange resins, deliquescent substances such as calcium chloride, and substances that adsorb water at the porous surface, such as aluminum oxide.

In the manufacturing method of the present embodiment, the hydrogen generating agent 14 is stored in the third container 13 together with the water 15. In this case, the hydrogen generating agent 14 may be directly stored in the third container 13, but the reaction heat generated when the hydrogen generating agent 14 reacts with the water 15 may possibly damage the third container 13. In a preferred embodiment, therefore, the hydrogen generating agent 14 may be, but is not limited to being, put into the third container 13 in a state in which the hydrogen generating agent 14 is enclosed in a bag body made of a film having hydrogen gas permeability, including nonwoven fabric. Details of the third container 13 will be described later. In an alternative embodiment, the hydrogen generating agent 14 may be used after being formed into one or more tablets, such as solid tablets.

The second container 12 may also have hydrogen gas permeability, but it may be equivalent to or lower than the hydrogen gas permeability of the first container 11. In a preferred embodiment, the second container 12 may be, but is not limited to, a container that includes a wrapping material using aluminum, such as an aluminum foil wrapping material and aluminum foil laminate film, and has gas barrier properties and flexibility. When the first container 11 is a glass container, the second container 12 may also be a glass container. The hydrogen permeability indicative of whether the hydrogen gas permeability is equivalent to or lower than that of an object may be measured as below. For example, with reference to a method as described in Patent Application No. 2009-221567, etc., a hydrogen-dissolved water that is stably maintained at about the saturated concentration (1.6 ppm under 20° C. and 1 atm) is generated to have a volume of 20 times the internal volume of a tightly-closed container as a measurement object, and the tightly-closed container filled fully with filtrated water (such as charcoal-treated water obtained by treating Fujisawa city tap water to pass through a charcoal column) is immersed in the hydrogen-dissolved water for five hours. Thereafter, the dissolved hydrogen concentration in the filtrated water is measured. When the dissolved hydrogen concentration is 1,000 ppb or lower, preferably 500 ppb or lower, more preferably 100 ppb or lower, and particularly preferably 10 ppb or lower, the container is deemed to be included in examples of the container of the present embodiment having equivalent or lower hydrogen permeability. As used herein, the term “equivalent or lower hydrogen permeability” and other similar terms have the same meaning as “hydrogen permeability equal to or lower than” that of an object.

The third container 13 is a container that can store the above-described hydrogen generating agent 14 and water 15 and allows the hydrogen gas generated in the container to be discharged to external. For example, as illustrated in FIG. 1(C), the third container 13 of the present embodiment comprises a container main body 131 and a cap 133 that has a duckbill valve 132. The container main body 131 is a bottom-closed tubular member formed of a heat-resistant resin material that can withstand the reaction heat of the hydrogen generating agent 14 and the water 15. After the water 15 is put into the container main body 131 and solidified (frozen), the hydrogen generating agent 14 is put into the container main body 131 and the cap 133 is fitted with an upper opening of the container main body 131. Like the container main body 131, the cap 133 is formed of a heat-resistant resin material that can withstand the reaction heat of the hydrogen generating agent 14 and the water 15. In the present embodiment, after the water 15 is put into the container main body 131 and solidified (frozen), the hydrogen generating agent 14 is put into the container main body 131. In an alternative embodiment, after water and the hydrogen generating agent 14 are put into the container main body 131 and rapidly solidified, the cap 133 may be fitted with the upper opening of the container main body 131.

The duckbill valve 132 is molded from a resin material having elasticity into a bird's bill-like shape and a slit is provided in the ridge line portion at the tip of the duckbill valve 132. According to this structure, the slit is in a state of being closed due to the own elasticity of the duckbill valve 132 until the inner pressure of the third container 13 becomes sufficiently high, and the sufficiently increased inner pressure then expands the slit of the duckbill valve 132 against the own elasticity to discharge the hydrogen gas. That is, the duckbill valve 132 is one example of a one-way valve or return check valve. In an alternative embodiment, a gas-permeable film may be provided as substitute for the duckbill valve 132 which is one example of the one-way valve or return check valve. The gas-permeable film allows the hydrogen gas generated in the third container 13 to be discharged to external of the third container 13.

After the hydrogen generating agent 14, the first container 11, the second container 12, and the third container 13 are prepared as the above, the liquid water 15 is stored in the container main body 131 of the third container 13 and solidified (frozen) as illustrated in FIG. 1(A), then the hydrogen generating agent 14 is stored in the container main body 131 of the third container 13 as illustrated in FIG. 1(B), and the cap 133 is fitted as illustrated in FIG. 1(C). Then, the first container 11 and the third container 13 are stored in the second container 12 as illustrated in FIG. 1(D) in a state in which the water 15 stored in the third container 13 is frozen, and the second container 12 is thereafter sealed as illustrated in FIG. 1(E).

Thus, the device 1 for generating hydrogen-containing liquid of the present embodiment is completed, but from when the second container 12 illustrated in FIG. 1(D) is sealed until when the liquid 16 in the first container 11 illustrated in FIG. 1(E) is used, the second container 12 which stores the first container 11 and the third container 13 is placed in an atmosphere that maintains the solidified state at least of the water 15 stored in the third container 13. For example, during the course of distribution and/or during the storage period after completing the manufacturing of the device 1 for generating hydrogen-containing liquid of the present embodiment, the device 1 for generating hydrogen-containing liquid is frozen and stored in order to maintain the solidified state of the water 15. This prevents the hydrogen generating agent 14 and the water 15 in the third container 13 from reacting with each other, and hydrogen gas is not generated.

When the liquid 16 in the first container 11 is used, the second container 12 which stores the first container 11 and the third container 13 as illustrated in FIG. 1(E) is placed in an atmosphere in which at least a part of the water 15 stored in the third container 13 melts. For example, the second container 12 which stores the first container 11 and the third container 13 as illustrated in FIG. 1(E) is moved from the freezing atmosphere to a room-temperature atmosphere of 0° C. or higher or, if the time to the use is short, heated to a temperature that is higher than room temperature and does not affect the liquid 16. This allows the melted water 15 and the hydrogen generating agent 14 to react with each other in the third container 13 to generate hydrogen gas therein, which is discharged into the second container 12 via the duckbill valve 132. As time passes, the hydrogen gas discharged into the second container 12 passes through the first container 11 so that hydrogen molecules are contained in the liquid 16 stored in the first container 11. When the liquid 16 is used, the second container 12 is opened and the first container 11 is taken out for the use.

Examples of a temperature higher than room temperature as referred to herein include a temperature that is not lower than 20° C. which is the “standard temperature” for storing medicines, preferably not lower than 25° C. which is the “upper limit of ordinary temperature,” more preferably not lower than 30° C. which is the “upper limit of room temperature,” and further preferably not lower than 40° C. which is the “upper limit of lukewarm temperature.”

The amount of the hydrogen generating agent 14 can be determined with reference to the criteria as below. For example, saturating normal saline solution in a bag of 500 mL volume (the normal saline solution corresponds to the liquid 16 and the bag corresponds to the first container 11) with hydrogen molecules under ordinary temperature/ordinary pressure (20° C., 1 atm) requires at least 0.4 mmol of hydrogen molecules (1.6 mg/L (solubility of hydrogen molecules)×0.5 L=0.8 mg=0.4 mmol). If metal calcium is used as the hydrogen generating agent 14 in this instance, at least 0.4 mmol=40.078 ×0.4=16.0312 mg of calcium is necessary in reference to the chemical reaction formula of metal calcium with water: Ca+2H₂O→Ca(OH)₂+H₂. However, if the bag (first container 11) has a margin of volume of 150 mL, the total volume as the sum of the volume and the margin is 650 mL, so 1.6 mg/L×0.65 L=1.04 mg=0.52 mmol can be obtained in accordance with the above calculation, that is, 0.52 mmol=40.078×0.52=20.84056 mg. It is therefore preferred that the hydrogen generating agent 14 be used with an amount of 20.84056 mg or more.

More in general, the substance quantity (mmol) of the hydrogen generating agent 14 is preferably not less than a value of (1.6 (mg/L)×(the fully filled volume (L) of the first container 11 having water hydrogen gas permeability and storing the liquid 16)/2)×((coefficient of the hydrogen generating agent in the left-hand side of the reaction formula of the hydrogen generating agent with water)/(coefficient of the hydrogen molecule in the right-hand side of the reaction formula of the hydrogen generating agent with water)), and may be more preferably not less than twice that value, more preferably not less than five times that value, and more preferably not less than ten times that value, in consideration of the margin ratio.

In order to obtain sufficient effects of hydrogen molecules on living organisms, the dissolved hydrogen concentration in a hydrogen-contained liquid may be 0.01 mg/L or more, preferably 0.05 mg/L or more, more preferably 0.1 mg/L or more, still more preferably 0.2 mg/L or more, yet more preferably 0.4 mg/L or more, further preferably 0.6 mg/L or more, still further preferably 0.8 mg/L or more, and yet further preferably 1.0 mg/L or more, when the liquid is used under a water temperature of 20° C. and 1 atm.

### Second Embodiment

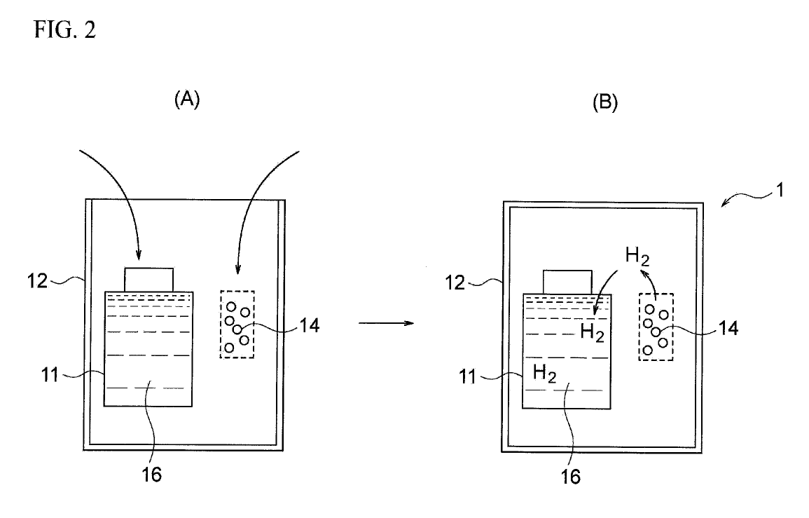

FIG. 2 is a flow diagram illustrating another embodiment of the method of generating hydrogen-containing liquid according to the present invention. This method includes preparing a hydrogen generating agent 14 that reacts with water to generate hydrogen gas, a first container 11 that encloses a liquid 16 applicable to a living organism and has hydrogen gas permeability, and a second container 12 that has hydrogen permeability equal to or lower than the hydrogen permeability of the first container 11. These first container 11, second container 12, hydrogen generating agent 14, and liquid 16 constitute a device 1 for generating hydrogen-containing liquid according to the present embodiment. Thus, the difference from the above-described first embodiment is that the third container 13 and water 15 are not prepared. Again, the third container 13 could store the hydrogen generating agent 14 and water 15 and discharge hydrogen gas generated inside to outside.

The type of the hydrogen generating agent 14, the type of the liquid 16, the structure of the first container 11, and the structure of the second container 12 are the same as those in the above-described first embodiment, and the description is borrowed herein.

After the hydrogen generating agent 14, the first container 11, and the second container 12 are prepared as the above, the first container 11 and the hydrogen generating agent 14 are stored in the second container 12 as illustrated in FIG. 2(A), and the second container 12 is then sealed. Then, the second container 12 which stores the first container 11 and the hydrogen generating agent 14 is frozen at a temperature of 0° C. or lower. Here, the hydrogen generating agent 14 to be stored in the second container 12 may be directly put into the second container 12 or may also be put into the second container 12 in a state in which the hydrogen generating agent 14 is enclosed in a bag body made of a film having hydrogen gas permeability, including nonwoven fabric. From when the second container 12 is sealed until when the liquid 16 is used, the second container 12 which stores the first container 11 and the hydrogen generating agent 14 is maintained in the frozen state. This prevents reaction of the hydrogen generating agent 14 in the third container 13, and hydrogen gas is not generated.

When the liquid 16 in the first container 11 is used, the second container 12 which stores the first container 11 and the hydrogen generating agent 14 is placed in an unfreezing atmosphere of 0° C. or higher as illustrated in FIG. 2(B). For example, the second container 12 which stores the first container 11 and the third container 13 is moved from the freezing atmosphere to a room-temperature atmosphere or, if the time to the use is short, heated to a temperature that is higher than room temperature and does not affect the liquid 16. This allows the water contained in the liquid 16 to evaporate in the second container 12, and the water vapor passing through the first container 11 reacts with the hydrogen generating agent 14 to generate hydrogen gas in the second container 12. In addition or alternatively, ice (originated from water in air) attached to the inner wall surface of the second container 12 due to the freezing process also melts, and this water 15 reacts with the hydrogen generating agent 14 to generate hydrogen gas in the second container 12. As time passes, the hydrogen gas generated in the second container 12 passes through the first container 11 so that hydrogen molecules are contained in the liquid 16 stored in the first container 11.

Examples of a temperature higher than room temperature as referred to herein include a temperature that is not lower than 20° C. which is the “standard temperature” for storing medicines, preferably not lower than 25° C. which is the “upper limit of ordinary temperature,” more preferably not lower than 30° C. which is the “upper limit of room temperature,” and further preferably not lower than 40° C. which is the “upper limit of lukewarm temperature.” In addition to the above, the second container 12 which stores the first container 11 and the hydrogen generating agent 14 may be maintained in a constant temperature reservoir of about 60° C. for 2 or 3 days.

### DESCRIPTION OF REFERENCE NUMERALS

1 Device for generating hydrogen-containing liquid
11 First container
12 Second container
13 Third container
   131 Container main body
   132 Duckbill valve
   133 Cap
14 Hydrogen generating agent
15 Water (Ice)
16 Liquid (Living organism applicable liquid)

What is claimed is:

1. A method of generating hydrogen-containing liquid, comprising:
preparing a hydrogen generating agent that reacts with water to generate hydrogen gas, a first container that encloses a liquid applicable to a living organism and has hydrogen gas permeability, and a second container that has hydrogen gas permeability equal to or lower than the hydrogen permeability of the first container;
storing the first container and the hydrogen generating agent in the second container and sealing the second container with no water except moisture in air;
maintaining the second container storing the first container and the hydrogen generating agent in a frozen state from when the second container is sealed until when the liquid is used;
upon use of the liquid, placing the second container storing the first container and the hydrogen generating agent in an unfreezing atmosphere so that the hydrogen generating agent reacts with water in the second container to generate hydrogen gas in the second container, the water in the second container including water vapor resulting from vaporization of water contained in the liquid stored in the first container and permeation of the water vapor through the first container into the second container and/or the moisture in air in the second container; and
allowing the hydrogen gas generated in the second container to pass through the first container so that hydrogen molecules are contained in the liquid stored in the first container.

2. The method of generating hydrogen-containing liquid according to claim 1, wherein placing the second container in the unfreezing atmosphere includes maintaining the second container in a temperature environment higher than room temperature for a certain period of time.

3. The method of generating hydrogen-containing liquid according to claim 2, wherein the temperature environment higher than room temperature is 30° C. or higher.
